(19) United States
(12) Patent Application Publication
(10) Pub. No.: US 2012/0197150 A1
(43) Pub. Date: Aug. 2, 2012

(54) EXTERNAL CARDIAC MONITOR

(75) Inventors: Jian Cao, Shoreview, MN (US); Christine Gale Kronich, Saint Paul, MN (US); David M. Fuss, Maple Grove, MN (US); Rebecca K. Gottlieb, Culver City, CA (US); Jeffrey L. Kehn, Chaska, MN (US); Paul Gordon Krause, Shoreview, MN (US); Brian B. Lee, Golden Valley, MN (US); George Patras, Greenfield, MN (US)

(73) Assignee: Medtronic, Inc., Minneapolis, MN (US)

(21) Appl. No.: 13/017,672

(22) Filed: Jan. 31, 2011

(51) Int. Cl. A61B 5/0402

(52) U.S. Cl. 600/523

(57) ABSTRACT

An external physiological monitor continuously senses and monitors cardiac function of a patient to allow detection of cardiac events and the recording of data and signals pre- and post-event. The monitor is connected to and suspended on the patient's body solely by the electrode assembly. Stored diagnostic data may be uplinked and evaluated by the patient's physician utilizing a programmer via a two-way telemetry link. An external patient activator may optionally allow the patient, or other care provider to manually activate the recording of diagnostic data.

### FIELD

The disclosure relates to a medical monitoring device. More particularly, the disclosure relates to a patient-worn electronic monitoring device.

### BACKGROUND

Syncopal events and arrhythmias of the heart are particularly problematic for diagnostic physicians to observe in living patients. These events can be of short duration and sudden onset, coming with little or no warning, and may happen very infrequently. Continuous cardiac monitoring of periods of time amounting to days or perhaps several weeks has been found useful for syncope diagnosis and AF monitoring. Many solutions to address the monitoring of these events have been proposed.

Implantable cardiac monitors such as Medtronic's Reveal® Insertable Cardiac Monitor are known for diagnosing the cause of recurrent, unexplained syncope or events possibly related to cardiac arrhythmias. The Medtronic approach is seen, for example, in the Klein et al. U.S. Pat. No. 5,987,352. However, the required minimally invasive surgical procedure can limit device usage among the patient population. Also, the relatively high costs associated with the device and implant procedure can limit device usage.

It is known that external cardiac monitors can have significant diagnostic yield in patients with frequent symptoms but user compliance is often poor due to the difficulty of portability. The external devices are generally hung on a belt, neck or shoulder strap, wrist worn, or carried by a patient using some other similar carrying arrangement. Sensors, such as ECG electrodes, are affixed to the patient's body, such as with tape, and connected to the battery operated monitor by wires. These external devices have been found to interfere with the patient's activities of daily living, making them impractical for long term use. Problems with external monitors and associated recorders also include inability of some patients to abide due to skin irritation, removal required for showering, and so on. Any time a living body needs to have long term monitoring of a physiologic event that is intermittent or infrequent or both, all these problems come into focus.

A recurrent problem with the conventional external cardiac monitors is that the electrode orientation on a patient is typically critical in ensuring proper device function. ECG mapping is typically required to determine optimal electrode placement for these monitors.

Another shortcoming of conventional external monitoring devices is that these devices lack the intelligence to vary the amount and type of data recorded. These medical monitors measure and record the full patient waveform, even when the patient is healthy. While transmitting the full patient waveform is the preferred solution from a purely clinical standpoint, such recordation requires significant amount of time to analyze the data to determine whether a cardiac condition exists. Also, the amount of device memory, device size and device battery limit the full waveform storage for longer recordings (e.g., a few weeks).

Accordingly, there still exists a need for a body worn recording and monitoring device having an external configuration and dimensions maximally adapted for enhanced patient compliance and suitable for long term use.

### SUMMARY

The inventors of the present disclosure have recognized that providing a device that enables coupling to a patient via the same electrodes that are conventionally utilized for monitoring or diagnosis of the patient is advantageous. For example, in contrast to the conventional wearable monitors, the inventors have described, in the present disclosure, devices that are constructed to be connectable and supportable on a patient's body via the electrodes thereby reducing interference with the patient's activities of daily living.

According to one aspect of the present disclosure, a body worn cardiac monitoring device includes an electronic package coupled to an electrode assembly for monitoring and recording a patient's electrocardiogram (ECG) signals. The electrode assembly includes a pair of electrodes that are directly non-permanently affixed to the skin surface of the patient with the entire cardiac monitoring device being secured to the patient only through the electrode contact. The electrode assembly includes a pair of medical electrical leads electrically coupling the electronic package with the electrodes. Each of the medical electrical leads connects to one of the pair of electrodes through a removable snap-fit connection. The cardiac monitoring device may include a hermetically sealed casing for housing the electronic package and the leads may be introduced into the casing through apertures on opposite ends of the housing.

According to another aspect, the body worn cardiac monitoring device is capable of communicating with an external device. The body worn cardiac monitoring device may include a short-range wireless transmitter to transmit the monitored or recorded signals to an external device such as a programmer. Other embodiments may include an external device for manually activating certain operations of the body worn cardiac monitoring device. In these cases, the external device includes a matched wireless receiver configured to receive the signals from the body worn cardiac monitoring device.

In other embodiments, the cardiac monitoring device has the capacity to use manual or automatic triggers or both to cause the memory to store events in reserved areas of a looping memory, preferably in identifiable memory partitions. It can accept limited programming or mode control and can read out sections of, or all of, the memory when prompted by a physician or other user, provided they have the appropriate device to initiate and receive such transmissions from the monitoring device.

In other embodiments, a system is provided for long term cardiac monitoring having a body worn device with the capacity for automatic triggering and manual triggering. A patient activator in communication with the body worn device may provide the manual trigger. A programmer is also provided for uplink and downlink communication with the body worn device.

### BRIEF DESCRIPTION OF THE DRAWINGS

FIG. 1 is a conceptual diagram illustrating an exemplary system that may be used to monitor one or more physiological parameters of patient.

FIG. 2 depicts an exploded perspective view of an exemplary external physiological monitor.

FIGS. 3A-C illustrate simplified schematic views of external physiological monitor as it would be used to obtain ECG signals from a patient.

FIG. 4 is a graph of time dependent ECG waveforms generated by an external physiological monitor in accordance with principles of this disclosure.

FIG. 5 depicts a fabrication process for an exemplary embodiment of an external physiological monitor constructed in accordance with principles of this disclosure.

### DETAILED DESCRIPTION

Embodiments of the present disclosure generally describe external physiologic monitoring devices that overcome the disadvantages of conventional external monitors. Prior to this disclosure, conventional body worn devices have generally fallen into two categories. The first category comprises devices that have electrodes that are fixedly positioned onto a housing with the entire housing being adhered to a surface of a patient's body. One such device is disclosed in the U.S. patent application No. 2009/0076345, by Manicka et al. Due to the obvious practical limitations relating to the size of devices in this category, only a limited number of electrode orientations can be attained. Therefore, it is generally required that an ECG mapping procedure be performed to determine appropriate electrode position and orientation prior to placing the device. It is often necessary that these measurements be made in several typical patient postures to account for posture variability as well.

The second category comprises devices that are generally carryable on a person. Examples of these devices are disclosed in U.S. Pat. Nos. 7,257,438, 7,680,523, and 7,630,756. Such devices typically require the use of a belt, lanyard, or strap for carrying the device often with a set of cables connecting the device to the electrodes. The cables can become tangled and cause discomfort or become unplugged when inadvertently pulled. In addition, wire motion can increase noise due to the triboelectric effect. Also, it is easily apparent that long term use of these devices is problematic and interferes with the patient's activities of daily living.

In contrast to the conventional wearable monitors, the present disclosure provides devices that are constructed to reduce interference with activities of daily living. In general, the present disclosure is related to devices for external monitoring of a patient's physiological parameters with the device being supported on the patient's body solely by the electrode assembly. In the following description, references are made to illustrative embodiments. The description is merely exemplary in nature and is not intended to limit the invention or the application and uses of the invention. Furthermore, there is no intention to be bound by any theory presented in the preceding background or the following detailed description. For purposes of clarity, the same reference numbers are used in the drawings to identify similar elements.

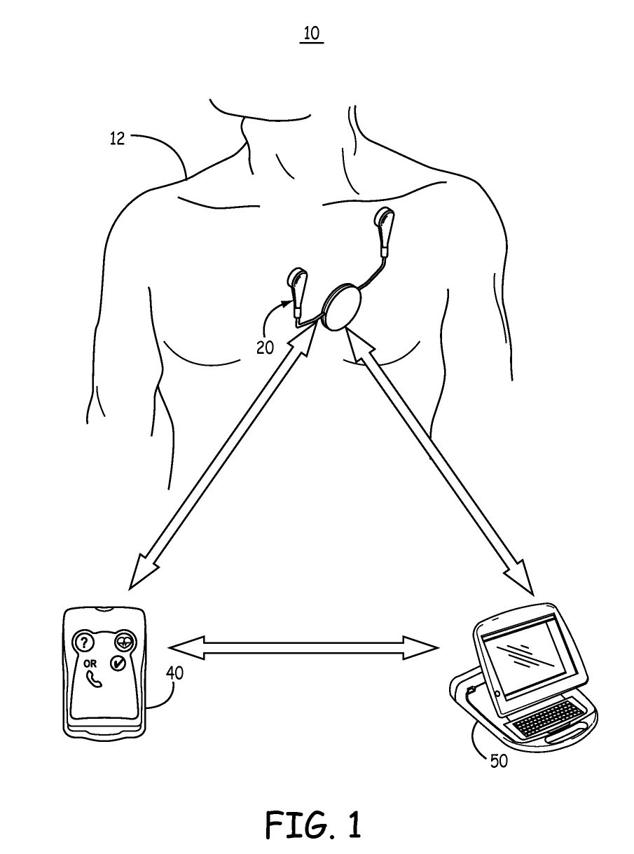

FIG. 1 is a conceptual diagram illustrating an example system 10 that may be used to monitor one or more physiological parameters of patient 12. Patient 12 ordinarily, but not necessarily, will be a human. System 10 includes an external physiological monitor (EPM) 20, a patient activator 40 and programmer 50. EPM 20 may be, for example, a cardiac monitor for monitoring electrocardiogram (ECG) signals, an electroencephalogram (EEG) monitor, a glucose monitor, a respiratory monitor and/or a device capable of external monitoring of physiological signals. For simplicity, however, operation of the EPM 20 will be described in relation to ECG signals.

The patient activator 40 may, in one embodiment, be a small handheld external device which may take any number of different forms. The patient activator 40 facilitates triggering of a preserved form of a recorded ECG signal. In one embodiment, patient activator 40 is a handheld battery-powered device which uses a coded radio-frequency telemetered signal to the EPM 20, on the press of a button. In other embodiments, the patient activator 40 interacts with the EPM 20 through a magnetic field such that holding the patient activator 40 adjacent to EPM 20 closes a magnetic switch within EPM 20 to trigger it.

In some examples, programmer 50 may be a handheld computing device or a computer workstation. Programmer 50 may include a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 50 can additionally or alternatively include a peripheral pointing device, such as a mouse, via which a user may interact with the user interface. In some embodiments, a display of programmer 50 may include a touch screen display, and a user may interact with programmer 50 via the display.

A user, such as a physician, technician, or other clinician, may interact with programmer 50 to communicate with EPM 20. For example, the user may interact with programmer 50 to retrieve the information from EPM 20. The retrieved information may include the rhythm of the patient's 12 heart, trends therein over time, or tachyarrhythmia episodes. As another example, the user may use programmer 50 to retrieve information from EPM 20 regarding other sensed physiological parameters of patient 12, such as intracardiac or intravascular pressure, activity, posture, respiration, or thoracic impedance. As another example, the user may use programmer 50 to retrieve information regarding the performance or integrity of EPM 20. A user may also interact with programmer 50 to send commands for programming EPM 20, e.g., select values for operational parameters of the EPM. In some examples, the user may activate certain features of EPM 20 by entering a single command via programmer 50, such as depression of a single key or combination of keys of a keypad or a single point-and-select action with a pointing device.

EPM 20 and programmer 50 may communicate via wireless communication using any techniques known in the art. Examples of communication techniques may include, for example, low frequency or radiofrequency (RF) telemetry, but other techniques are also contemplated. In some examples, programmer 50 may include a programming head that may be placed proximate to the patient's body near the EPM 20 in order to improve the quality or security of communication between EPM 20 and programmer 50.

Other methods for triggering ECG data retention in memory (each of which has its own advantages for implementation) are to use physical tapping or slapping of the finger or hand on the skin over the device in a particular cadence and/or number of taps the advantage being that no triggering device is needed. A microphone receiver may also be built into the EPM 20 to enable matched voice activation with a known command to be suitably employed. Another approach is light activation using a light source and receiver. Any or all of these methods of patient activation may be employed in conjunction with an automatic activation or trigger for holding a chunk of memory. This could be activated by automatic recognition of an arrhythmia, a heartbeat that is too fast or too slow, or for any other condition the device may be set up to find.

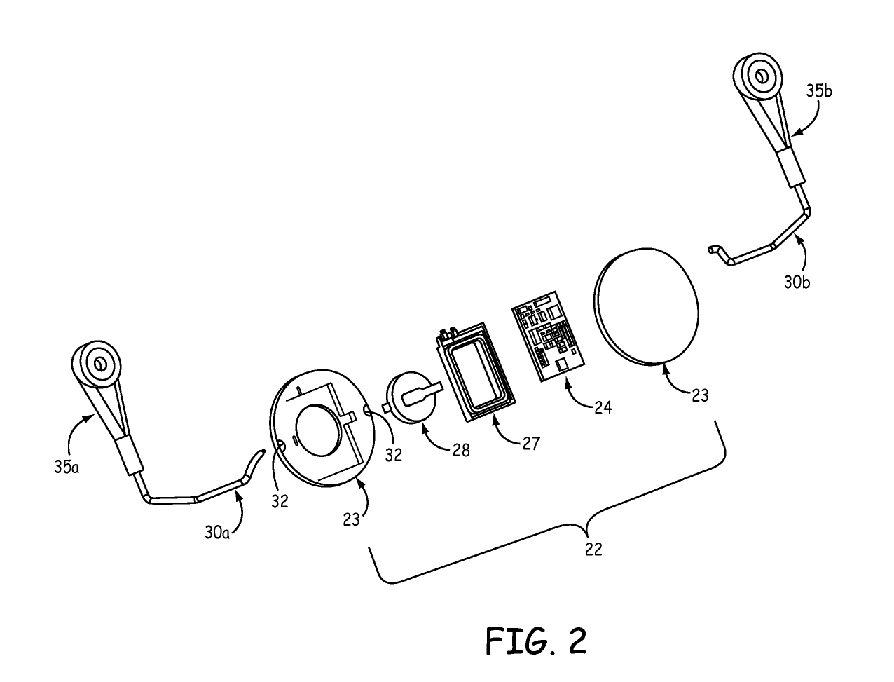

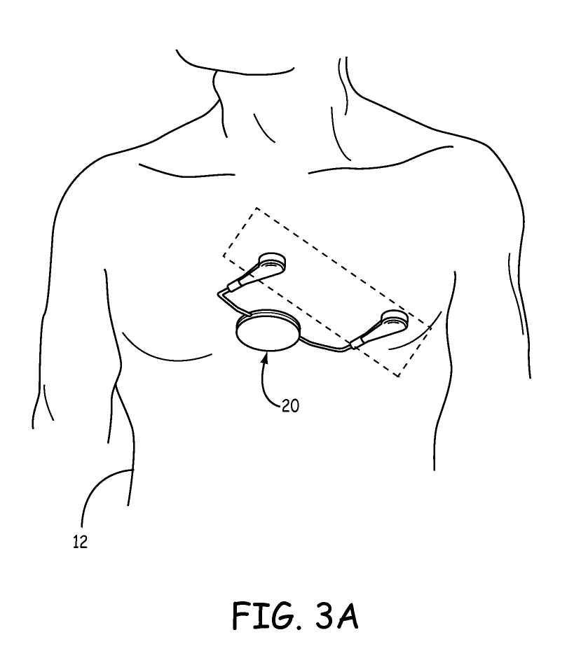

FIG. 2 depicts an exploded perspective view of one exemplary external physiological monitor (EPM) 20. The EPM 20 comprises an electronics package 22 that includes a housing 23 and circuitry assembly 24 for monitoring a patient's ECG, loop recording the monitored ECG and selectively storing portions of the recorded ECG data for retrieval by an external user device. Housing 23 may optionally include a button 31 electrically coupled to circuitry assembly 24 for activating the loop recording. The button 31 may be activated by the patient 12 to manually activate diagnostic data recording in instances, for example, when the patient 12 feels an onset of symptoms that may be related to a cardiac event.

Housing 23 comprises a shield set, or two shell casing, that is configured to enclose the electronic components. The shield set of housing 23 may provide a hermetic enclosure although some embodiments may simply provide for the enclosure to be water tight. For example, the housing 23 may be constructed from biocompatible plastic material such as Polycarbonate and fabricated utilizing an injection molding process. By providing a hermetic or water tight enclosure, the patient may proceed with activities of daily living such as taking showers or even exercise that can result in sweating without concern about adversely affecting device functionality. While not intended to be limiting, the illustrative embodiment discloses a circular shaped housing 23. The housing 23 has dimensions of about 1.5 inches in diameter with a thickness of about 0.5 inches. Nevertheless, the housing 23 may be configured in any other desired geometrical shape, or otherwise, including a square shape, rectangular shape, a hexagon shape, a pentagon, etc.

The circuitry assembly 24 associated with EPM 20 may correspond to that described in conjunction with any of the various embodiments shown in U.S. Pat. No. 5,987,352 “Minimally Invasive Implantable Device for Monitoring Physiologic Events” to Klein, et al., incorporated herein by reference in its entirety. Briefly, the circuitry assembly 24 contains an amplifier, memory, microprocessor, receiver, transmitter and other electronic components (generally, “electronics 25”) required for the device function and a telemetry antenna 26 to communicate data from the EPM 20. Programming of the device or mode setting will also use the telemetry antenna 26 and associated circuitry. The electronics 25 includes circuitry and intelligence required for the device function and a memory component for storing data and commands. Circuitry assembly 24 is electrically coupled to a power source such as battery 28. The battery 28 may be a lithium coin cell, such as the standard type CR2032, produced by numerous manufactures or a rechargeable battery.

Electronics package 22 may also be furnished with various sensors (not shown), in addition to the customary signal processing and related electronics. For example, an accelerometer and inclinometer may be provided to detect activity and posture of the patient, providing useful information for correlation with the other vital signs.

A pair of leads 30a-b is provided for coupling the electronic package 22 to a pair of electrodes 35a-b. Together, the leads 30a-b and electrodes 35a-b form an electrode assembly. The leads 30a-b may comprise an external resilient substrate that is suitably selected for encasing one or more conductors. The substrate material is selected to permit flexing in a complimentary manner in response to a patient's body movements to provide for patient comfort and wearability. One end of each of the leads 30a-b may be permanently electrically coupled to the electronic package 22 with the other of the free ends being configured for snap-fit coupling with the electrodes 35a-b.

One of the primary challenges in the detection of the surface ECG signals is their relatively small amplitudes. Additionally, these low amplitude signals are more susceptible to being masked and/or distorted by the electrical noise produced by a moving body, the aforementioned triboelectric effect, as well as the noise produced by the device itself. Noise, in this context, refers to both contact noise created by such movement and interaction of the body and device, as well as electronic noise detected as part of the signal reaching the sensors. An important consideration for eliminating noise is the length of leads 30a-b and their attachment onto the housing 23. Each of the leads 30a-b may have a length of about 1.25 inches to about 2 inches. An example of such a lead may be the 5LD Polyurethane leads manufactured by Rozzin Electronics, Inc.

Additionally, embodiments of the present disclosure include arrangements of leads 30a-b that prevent entanglement. This may be achieved by positioning the leads 30a-b on opposite ends of the housing 23 or by incorporating a separator (not shown) to force the separation of the lead pair. Accordingly, housing 23 may be formed with a pair of apertures 32 through which the leads 30a-b are inserted for coupling to the electronic package 22. However, such an implementation is merely illustrative. For example, those skilled in the art are familiar with various feedthrough assemblies that may suitably be molded into the housing 23 and utilized in the coupling of leads 30a-b to the electronic package 22. In the illustrative embodiment the apertures 32 are configured to be located at points that are substantially diametrically-opposed on the longitudinal axis of the housing 23. As such, the location of the apertures 32 facilitates a lead attachment that facilitates reduction of noise and prevents lead wire entanglement.

Electrodes 35a-b may be selected from any suitable surface ECG electrodes. As previously stated, one embodiment of the disclosure provides for snap-fit engagement of electrodes 35a-b to the leads 30a-b. This configuration permits the assembly of the electronic package 22 and leads 30a-b to be coupled and decoupled from the electrodes 35a-b, as desired, without compromising functionality. As such, the electrodes 35a-b may be swapped out when necessary with a new pair, without requiring an entirely new EPM 20. Thus, the lead-to-electrode snap-fit coupling is advantageous in that it permits the EPM 20 to be used over extended periods of time and/or by multiple users with the simple change of electrodes. The snap-fit engagement also permits a patient to easily perform the coupling and decoupling without requiring a skilled technician to perform the procedure.

In accordance with aspects of this disclosure, the EPM 20 is held onto the surface of a patient with the electrodes 35a-b. In one embodiment, the electrodes 35a-b may be constructed in accordance with the general teachings of U.S. Pat. No. 4,681,118, “Waterproof electrode assembly with transmitter for recording electrocardiogram” to Asai et al., incorporated herein by reference in its entirety. Other suitable electrodes include the hypoallergenic Ambu® Blue Sensor VLC manufactured by AMBU, or Red Dot™ adhesive electrodes sold by 3M, which are disposable, one-time use electrodes, or known reusable electrodes made of, for example, stainless steel, conductive carbonized rubber, or some other conductive substrate, such as certain products from Advanced Bioelectric in Canada.

It should be noted that some of the existing electrodes typically have higher coupling impedances that can impact the performance of the electrodes. Thus, to counteract the impedance effects, the electrodes 35a-b may include a gel-backed surface that contains an adhesive for coupling to the patient 12. In such a configuration, the entire skin contacting side of the electrodes 35a-b may be coated with a conductive adhesive gel or lotion. Suitable gels include the Buh-Bump, manufactured by Get Rhythm, Inc. of Jersey City, N.J. This conductive adhesive gel acts as an electrolyte between the contact area of the electrodes 35a-b and the patient's skin surface. Optionally, the electrode 35a-b may be covered with a protective release paper that releasably covers the surface of the electrode 35a-b to protect the electrodes and the adhesives. In alternative embodiments, the electrodes 35a-b may be provided with a plurality of microneedles for, among other things, enhancing electrical contact with the skin and providing real time access to interstitial fluid in and below the epidermis.

One important consideration in accordance with aspects of the present disclosure relates to the weight and dimensions of the electronic package 22. This is especially true in light of the fact that the EPM 20 is configured to be held in place solely by the adhesion of electrodes 35a-b on the patient's skin. While it is not the intent of the inventors of the present disclosure to detail in this disclosure the individual selection of constituent components of the entire EPM 20, it is believed sufficient to observe that the components of electronic package 22, for example, were selected such that the weight of the package 22 was limited to approximately 9 grams.

Optionally, a surface of the housing 23 may include an adhesive portion that can facilitate adhesion of EPM 20 to the patient. However, this option may not be desirable because a need may arise for the patient to decouple the EPM 20, such as, for purposes of changing the power source.

As seen from the described embodiment, EPM 20 continuously senses and monitors cardiac function of the patient 12 via the electrodes 35a-b located on the patient's surface to allow detection of cardiac events and the recording of data and signals pre- and post-event. The EPM 20 may include a manual activation mode in which the patient provides an indication (e.g., push a button on the EPM 20, patient activator 40 or programmer 50) when a cardiac event is occurring or has just occurred. In the manual activation mode, to allow for the fact that the patient may not mark the cardiac event until the cardiac event is in progress or has ended, the ECG loop recording may begin a longer time period before the event is marked. For example, the medical device system may save ECG data beginning 15 minutes before the patient mark. This time period may be programmable. Post-processing of this saved signal will analyze the data to evaluate heart rate changes during the cardiac event, heart rate variability and changes in ECG waveforms. Generally speaking, the specifics of the manual activation by the patient (or caregiver) will involve pushing a button on the EPM 20, patient activator 40 or programmer 50. This will provide a marker and will initiate a loop recording. In addition, prolonged ECG loop recordings are possible (e.g., in the case of SUDEP, recording all data during sleep since the incidence of SUDEP is highest in patients during sleep).

An arrhythmia detector such as that disclosed in the aforementioned '352 patent may also be included for automatic activation or to trigger the holding of a chunk of memory. The arrhythmia detector analyzes the monitored ECG data to detect the occurrence of an arrhythmia event afflicting a patient's heart. Upon automatically recognizing an arrhythmia, a heartbeat too fast or too slow, or any other condition the device may be set up to find, the arrhythmia detector supplies an automatic trigger signal for initiating a loop recording. The arrhythmia detector therefore provides the capability to maintain a data record over a long period of time as well as highlighting or at least capturing those physiologic events that are of interest to a diagnostic, research or therapeutic study, and particularly those physiologic events that are required for correct diagnosis and therapy. The stored or recorded diagnostic data is uplinked and evaluated by the patient's physician utilizing programmer 50 via a two-way telemetry link.

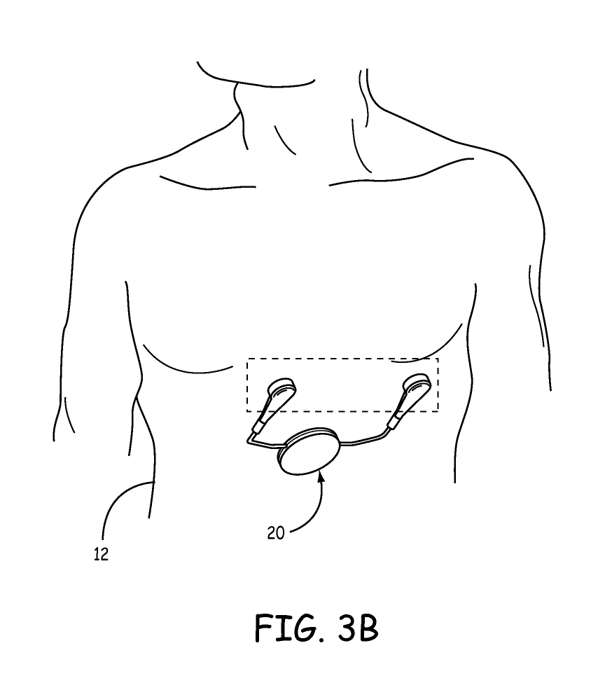

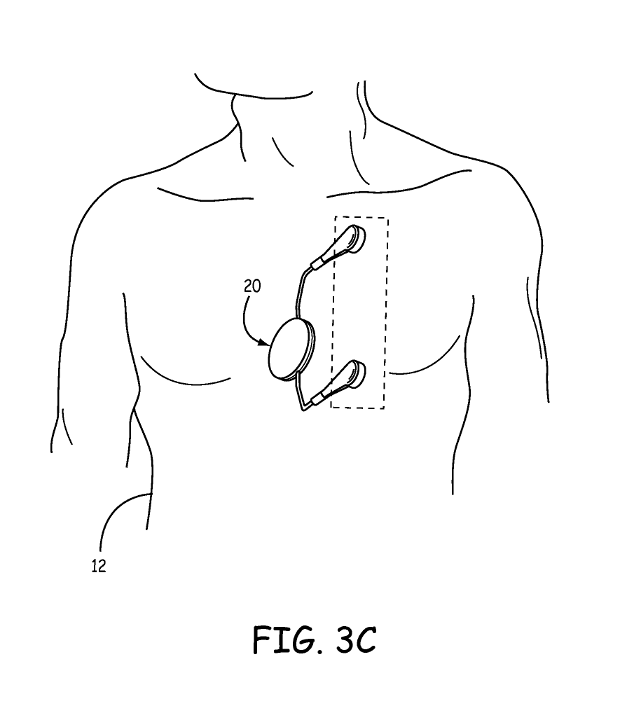

FIGS. 3A-C illustrate simplified schematic views of EPM 20 as it would be used to obtain ECG signals from patient 12. The human heart is a source of a voltage potential difference resulting from the electrical activity that causes the heart muscles to contract. This potential difference is known in the art as the heart's action potential. An ECG signal is a measurement of this action potential. As those skilled in the art may know, the heart's electrical activity can be modeled as an electric dipole, which varies both in orientation and amplitude over the cardiac cycle. Such a dipole introduces electric field lines connecting its endpoints in the surrounding media. It is these field lines that give rise to the potentials observed at surface electrodes. The potential developed is dependent on the strength of the field, the separation of the electrodes and the angle between the axis of the electrodes and the field lines. The potential is ideally greatest when the field is parallel to the electrode axis and zero when orthogonal. It is for this reason that different electrode orientations produce differing ECG waveform morphologies, since the relative orientations of the electrode axis and the electric field corresponding to a particular feature of the waveform will dictate the amplitude and polarity with which that feature appears on the waveform.

Conventional ECG electrode placements have been selected with the intention of providing useful and informative “views” of the heart's electrical activity during the various phases of the cardiac cycle. Due to the diminished amplitude of the ECG signal on the surface of the body, as well as the attendant noise resulting from among other things the patient's own motion, it is generally desirable to perform tests such as ECG mapping to determine the optimal locations for monitoring the surface ECG signals with conventional devices. Note that, according to well-known principles of field mapping, the electric field lines permeate the medium surrounding the dipole causing them. Although the strongest signals may be obtained with electrodes located near the ends of the dipole, weaker signals are obtained at other locations, including even when the dipole does not lie between the electrodes. There are, of course, cases when no signal is obtained, such as in the case of orthogonality, or when some distortion of the field prevents the field lines from reaching the electrode site. Therefore, the actual body surface potentials are considerably distorted from those that would ideally exist were the heart's electric dipole to induce its electric field lines in a homogenous, infinite medium.

Electrodes are conventionally placed in two different quadrants on the body, where the body is divided into four sections, or quadrants, by two planes running through the heart. The location of these planes has been modified over time as knowledge in this field has progressed, but has remained fairly constant in that a sagittal plane runs roughly vertically through the heart and a transverse plane runs roughly horizontally. These two planes are orthogonal to one another when viewed from the two-dimensional perspective from in front of the patient. The assessment of these imaginary planes is believed inconsequential for applications utilizing the EPMs of this disclosure.

In comparison to conventional devices, the present disclosure allows for more optimal placement of the electrodes because of the electrode spacing afforded by the assembly of the leads 30a-b and electrodes 35a-b. Specifically, the flexibility provided by the leads 30a-b allows the electrodes to 35a-b to be separated by a sufficient length, typically in the range of about 3 inches to about 5 inches. The relatively large length will allow for a sufficiently large QRS complex for sensing, accurate automatic detection of arrhythmias and better p-wave visibility for rhythm diagnosis. In addition, no specific mapping is required in order to get good signals in a specific region (left heart areas). In one embodiment, the length of leads 30a-b is selected such that the spacing between electrode 35a and electrode 35b is 4 inches. The configuration of EPM 20 facilitates placement of the electrodes 35a-b with enough separation between them to detect the action potential signals and therefore achieve optimal R-wave sensing performance.

Turning then to FIGS. 3A-C, the connection of EPM 20 to patient 12 is achieved through the electrodes 35a-b. In other words, the assembly of electrodes 35a-b to leads 30a-b is the sole means by which the electronics package 20 is suspended on the patient 12. In FIG. 3A the electrodes 35a-b are illustrated as being oriented in an orthogonal direction in relation to the left and right chambers of the patient's heart. In this configuration, it should be noted that the electronics package 20 is suspended on the patient's body primarily with the electrode 35a when the patient is upright.

In FIG. 3B patient 12 is shown having electrodes 35a-b suspending the EPM 20 in a perpendicular orientation in relation to an imaginary vertical axis defined by the top-to-apex of the heart. This electrode orientation permits optimal sensing of the surface potentials corresponding to the heart's R-waves.

FIG. 3C illustrates the electrodes 35a-b placed in a direction that is generally parallel to the imaginary vertical axis defined by the top-to-apex of the patient's heart. This orientation is ideal for monitoring and distinguishing atrial and ventricular events. Like the configurations depicted in FIGS. 3A and 3B, the construction of the EPM 20 of FIG. 3C provides an electrode spacing that will suitably permit potentials associated with the atrial and ventricular events to be distinguished from other signals such as noise. This is because the electrode spacing makes it unlikely that noise occurring at one of the electrodes 35a and 35b can occur simultaneously at both electrode sites. Also, like the configuration of FIG. 3A, the electronics package 20 is suspended on the patient's body primarily with the electrode 35a when the patient is upright.

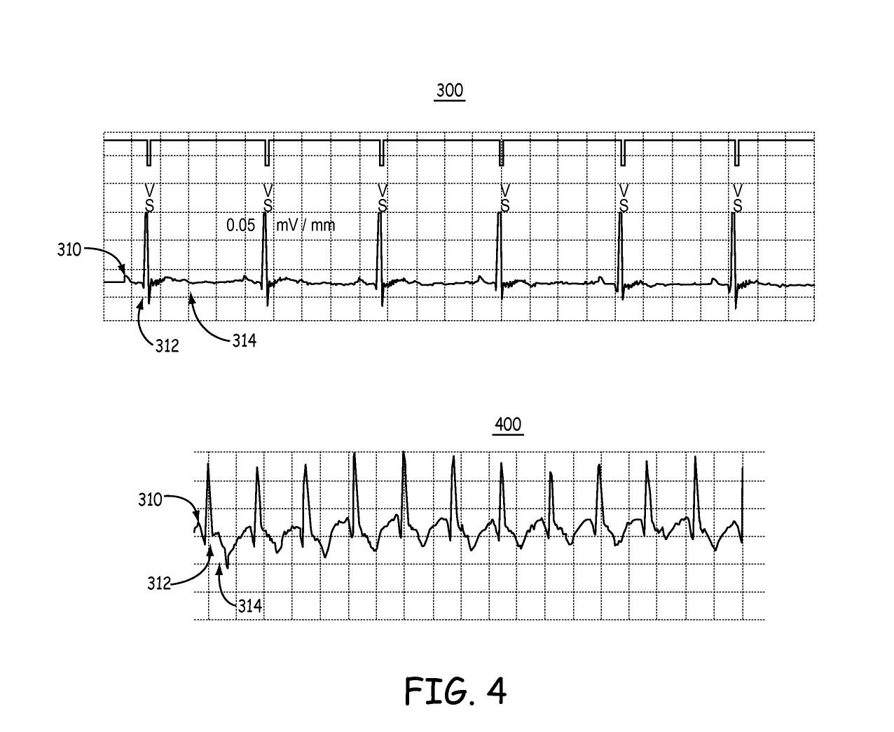

FIG. 4 is a graph of time dependent ECG waveforms 300, 400 generated by an external physiological monitor in accordance with principles of this disclosure. The signal strength of the ECG waveforms is shown on the Y axis and time is shown on the X axis. Generally speaking, the individual spikes and dips in the waveforms 300, 400 are called waves. The P wave 310 represents the contraction of the atria, while the Q, R, and S waves, referred to as the QRS complex 312, represent the contraction of the ventricles. The T wave 314 represents the recovery, or repolarization, of the ventricles. The amplitude of a typical ECG signal was found to be approximately 0.3 to 5 mV when measured from the patient's body in accordance with embodiments of the present disclosure. Following a heartbeat, the electrical impulse travels essentially instantaneously from the patient's heart, where the electrodes 35a-b detect it to generate the ECG waveform.

An EPM 20 that was attached to a patient in the orientation illustrated in FIG. 3C was used to record the ECG waveform 300. The ECG waveform 300 is a typical electrocardiogram signal characterized by a repeating pattern of several distinct segments, including a P wave 310, a QRS complex 312, and a T-wave 314. These are all shown as portions of a solid line in FIG. 4 and are discussed more fully below.

The ECG waveform 400 was obtained from a patient with an EPM suspended on the patient in the orientation generally illustrated in FIG. 3A. The various portions of the electrocardiogram are shown as the P, QRS complex, and T waves. Here again, these are all shown as portions of a solid line in FIG. 4. The ECG waveform 400 is characterized by a repeating pattern of several distinct segments, including QRS complex 312. The QRS complex 312 comprises a peak 404. The time interval between two consecutive peaks 404 is the interbeat interval 406 (“RR interval”). The peak 404 is one of the QRS 312 features which can be used to detect a QRS complex. As those skilled in the art can appreciate, the instantaneous heart rate is the inverse of the RR interval 406, that is, instantaneous heart rate equals 1/RR, for each RR interval 406. These RR intervals or the instantaneous heart rate can be used to detect fibrillation in conjunction with the aforementioned arrhythmia detector using any known techniques.

For example, the RR interval 406 (beat-to-beat) variation of heart rate is computed using the absolute value of the difference of each RR interval 406 heart rate from the local mean, which is the mean value for a selected number of RR intervals 406 used for the computation. Each RR interval 406 is used to compute the instantaneous heart rate for the RR interval 406. The sequence of these instantaneous heart rates for each RR interval 406 can be used for detecting atrial fibrillation. The heart rates obtained are not averaged over fixed time intervals, thus avoiding loss of variability data over the fixed time intervals. When an arrhythmia occurs, there is typically a decrease in the RR interval, meaning that the R-waves occur more frequently per period of time. It is this decrease in the RR interval as shown in waveform 400 compared to the waveform 300 that is an indicator that an arrhythmia has occurred.

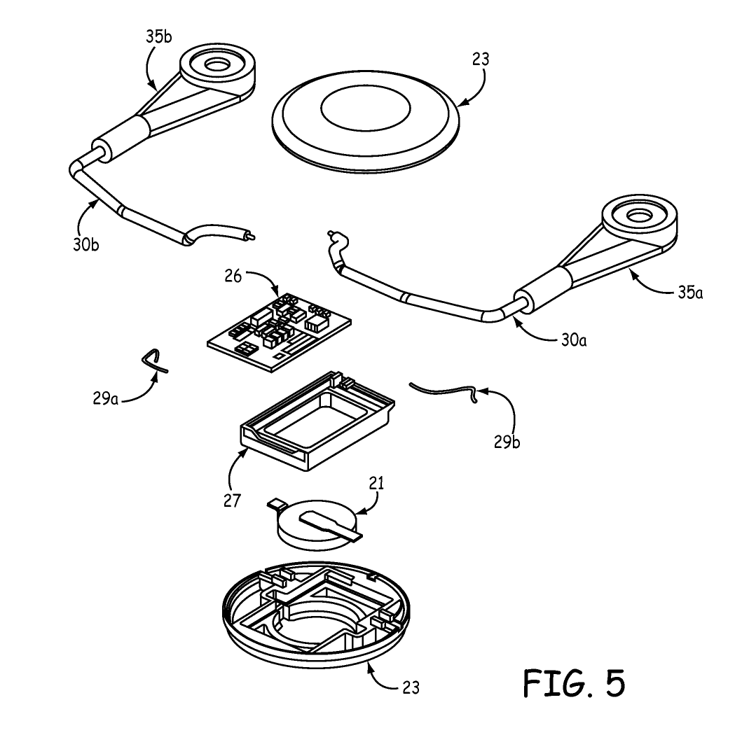

FIG. 5 depicts a fabrication process for an exemplary embodiment of an external physiological monitor constructed in accordance with principles of this disclosure. First an optional circuitry assembly cup 27 may be provided for fixedly mounting the circuitry assembly 24 within the housing 23. An adhesive compound is applied to the interior surface of the circuitry assembly cup 27 and the circuitry assembly 24 is positioned within the cup 27. The adhesive compound may be a medical grade UV adhesive such as Ultra-Red™ 1120-M-UR manufactured by DYMAX. The telemetry antenna 26 is then soldered to the circuitry assembly 24. Two battery wires 29a-b for coupling battery 28 to the circuitry assembly 24 are soldered onto a battery holder 21 with the battery holder 21 being mounted onto the housing 23 bottom shell casing. Battery 28 is then installed onto the battery holder 21. Any suitable battery 28 such as the Energizer CR1632 button cell may be employed. Subsequently, an adhesive compound such as the above referenced, Ultra-Red adhesive, is applied to the bottom exterior surface of the circuitry assembly cup 27 and the cup 27 is then placed within a preformed portion of the housing 23 bottom shell casing. In one embodiment, the assembly cup 27 may include battery terminals (not shown) that electrically couple the battery wires 29a-b to the circuitry assembly 24 upon placement of the circuitry assembly 24 into the housing 23 without requiring soldering. Next, the lead pair 30a-b is placed in the channels or apertures 32 provided in the bottom shell casing of housing 23. The distal ends of each of the lead pair 30a-b are soldered onto the circuitry assembly 24. In the illustrative embodiment, electrodes 35a-b may be the aforementioned snap electrodes and are pre-assembled onto the lead pair 30a-b. Finally, the top shell casing of housing 23 is coupled to the bottom shell casing by, for example, application of an adhesive on the outer circumference edges.

Thus, various examples for external monitoring of physiological parameters have been presented in the foregoing description with reference to specific embodiments. It is appreciated that various modifications to the referenced embodiments may be made without departing from the scope of the disclosure as set forth in the following claims.

What is claimed is:

1. A cardiac monitoring apparatus, comprising:
an electronic package including:
a housing having a longitudinal dimension exceeding a transverse dimension;
electronic circuitry disposed within the housing for monitoring and recording a patient's electrocardiographic (ECG) signals;
an electrode assembly coupled to the electronic package, the electrode assembly including:
a first medical electrical lead having a first end coupled to the electronic circuitry and a second end coupled to a first conductive electrode; and
a second medical electrical lead having a first end coupled to the electronic circuitry and a second end coupled to a second conductive electrode,
wherein the first and second conductive electrodes are configured for connection to a surface on the patient's body and the electronic package is dimensioned to be suspended on the patient's body via the connection of at least the first electrode.

2. The cardiac monitoring apparatus of claim 1, wherein the housing includes first and second apertures positioned on opposing ends of the housing along the transverse dimension, with the first and second ends of the first and second medical electrical leads being inserted through the first and second apertures, respectively, of said housing.

3. The cardiac monitoring apparatus of claim 1, wherein the electronic package is suspended on the patient's body by the connection of the first and second conductive electrodes on the surface of the patient.

4. The cardiac monitoring apparatus of claim 1, wherein the electrode assembly is configured to provide a separation between the first and second conductive electrodes of about 4 inches.

5. The cardiac monitoring apparatus of claim 1, wherein the housing has a length in the range of about 1 inch to about 2 inches.

6. The cardiac monitoring apparatus of claim 1, wherein the electronic package has a weight of approximately 9 grams.

7. The cardiac monitoring apparatus of claim 1, wherein the housing comprises a generally circular configuration and the first and second apertures are disposed on diametrically opposed ends of the housing.

8. The cardiac monitoring apparatus of claim 1, wherein the housing comprises spaced apart first and second major side walls on the longitudinal dimension extending to and meeting with opposed third and fourth end walls on the transverse dimension.

9. The cardiac monitoring apparatus of claim 8, wherein the first and second major side walls define a rectangular configuration.

10. The cardiac monitoring apparatus of claim 1, wherein the first and second leads are dimensioned to each have a length in the range of about 1.25 inches.

11. The cardiac monitoring apparatus of claim 1, wherein the first and second conductive electrodes are connected to the patient's surface in an orientation that is generally aligned with an imaginary vertical axis of the heart.

12. The cardiac monitoring apparatus of claim 11, wherein the vertical axis is generally defined by an imaginary line extending from the right atrium to the left ventricle.

13. The cardiac monitoring apparatus of claim 1, wherein the first and second conductive electrodes are connected to the patient's surface in an orientation that is generally aligned with an imaginary horizontal axis of the heart.

14. The cardiac monitoring apparatus of claim 13, wherein the horizontal axis is generally defined by an imaginary line extending from the right ventricle to the left ventricle.

15. The cardiac monitoring apparatus of claim 1, wherein the monitoring and recording of the patient's ECG signals is activated in response to a user initiated trigger.

16. The cardiac monitoring apparatus of claim 1, further comprising an adhesive agent disposed on the housing for selectively connecting the housing to a surface of the patient.
